(19) United States
(12) Patent Application Publication
(10) Pub. No.: US 2004/0176841 A1
(43) Pub. Date: Sep. 9, 2004

(54) Medical prosthesis

(76) Inventor: Terrell W. Ferguson, Georgetown, KY (US)

Correspondence Address: KING & SCHICKLI, PLLC, 247 NORTH BROADWAY, LEXINGTON, KY, 40507, US

(21) Appl. No.: 10/480,400

(22) PCT Filed: Aug. 23, 2002
(86) PCT No.: PCT/US02/27072

Related U.S. Application Data
(60) Provisional application No. 60/314,988, filed on Aug. 24, 2001.
(60) Provisional application No. 60/344,164, filed on Dec. 27, 2001.
(60) Provisional application No. 60/351,783, filed on Jan. 25, 2002.

(51) Int. Cl. A61F 2/52

(52) U.S. Cl. 623/7; 623/926; 264/DIG. 30

(57) ABSTRACT

A medical prosthesis includes a body having a silicone skin and a silicone foam body core. The skin and foam body core are both resilient. The core includes a plurality of voids and a plurality of discontinuities. Some of the discontinuities communicate with more than one void.

This patent application claims the benefit of U.S. Provisional Patent Applications Serial No. 60/314,988 filed on Aug. 24, 2001, 60/344,164 filed on Dec. 27, 2001, and 60/351,783 filed on Jan. 25, 2002.

### TECHNICAL FIELD

The present invention relates generally to the field of medicine and more particularly to a medical prosthesis of a breast, nose, ear, digit, limb or other body part.

### BACKGROUND OF THE INVENTION

The making of medical prostheses for patients who have lost or damaged body parts is well known in the art. For example, U.S. Pat. No. 4,401,492 to Pfrommer discloses a breast prosthesis which is filled with a silicone gel. Such prostheses are generally heavy and uncomfortable for long-term wear. They also do not generally have life-like tactile qualities and, accordingly, a need clearly exists for more life-like medical prostheses.

### SUMMARY OF THE INVENTION

In accordance with the purposes of the present invention as described herein, an improved medical prosthesis is provided. That medical prosthesis comprises a body having a silicone skin and a silicone foam body core. More specifically describing the invention, the medical prosthesis includes a body having a silicone skin between about 1.0 to about 2.0 mm thick and a silicone foam body core having from about 25 to 41% by volume silicone and from about 75 to about 59% by volume void space. Thus, the silicone foam body core may be about, for example, 33% by volume silicone and about 67% by volume void space.

Still further, the silicone foam body core includes a plurality of voids where each void has a volume between about 0.06545 to about 268.0832 mm³. More typically, the silicone foam body core includes a plurality of voids wherein each void has a volume between about 65.45 and about 179.5948 mm³. The silicone foam also has a density of between 5.0 to about 10.0 lbs/ft³. Still more specifically describing the invention, the skin has a viscosity of about 50,000±10 CPS, a specific gravity of between about 1.0 and 1.01, a hardness shore A of between about 8 and about 12, a tear resistance of about 120 ppi, a tensile strength of about 475 psi, and elongation of between about 550 to about 600% and a shrinkage of about 0.1%. Still further, the silicone foam has a compression deflection at 25% psi of between about 0.5 to about 1.0 and at 50% psi of between about 0.5 to about 3.0 and a specific gravity of between about 0.1 and about 0.15.

In accordance with yet another aspect of the present invention, a breast prosthesis is provided comprising a body having a silicone skin and a silicone foam body core. The silicone skin and silicone foam body core of the breast prosthesis have the same physical characteristics and properties previously described above with respect to the more broadly identified medical prosthesis.

Further describing the breast prosthesis, a nipple of silicone is provided. The nipple is between about 2 to about 5 mm thick. Further, the breast prosthesis includes a peripheral margin of silicone from about 1.0 to about 2.0 mm thick and from about 9.5 to about 19.1 mm wide. Further, the breast prosthesis includes a rear surface having a profile matching a chest wall of a patient for which the breast prosthesis is made. This rear wall is formed from barrier silicone and is about 1.0 to about 2.0 mm thick.

In accordance with yet another aspect of the present invention, a method of making a breast prosthesis for a patient is provided. The method may be broadly defined as including the steps of (1) making a mold of appropriate shape for the breast prosthesis, (2) forming a nipple in the mold, (3) forming a skin surface and mounting rim on the mold, (4) forming a foam body core in the mold in contact with the skin surface and the mounting rim and (5) removing the breast prosthesis from the mold.

The method may be further defined as including using silicone to form the nipple, the skin surface and mounting rim and using silicone foam to form the foam body core. In addition, the method may include the step of forming the nipple, skin surface and mounting rim by applying silicone on the mold.

Still further, the forming of the foam body core includes the step of putting a mixture of silicone, a polymerization catalyst and a mechanical foaming agent into the mold. In addition, the method includes mixing the silicone, the polymerization catalyst and the mechanical foaming agent together in an amount of about 1 part by volume silicone foam and about 2 parts by volume mechanical foaming agent.

In accordance with still another aspect of the present invention, the method includes the employing of solid bodies that are insoluble in silicone as the mechanical foaming agent. The individual solid bodies utilized have a volume of between about 0.06545 to about 268.0832 mm³ and more typically have a volume of between about 65.45 to about 179.5948 mm³. The method further includes a step of removing the mechanical foaming agent from the cured breast prosthesis. Removing may be accomplished in a number of ways including by pressing the mechanical foaming agent from the breast prosthesis, vacuuming the mechanical foaming agent from the breast prosthesis and applying a magnetic field to draw the mechanical foaming agent from the breast prosthesis. For certain applications and in certain situations, the method may also include the step of lubricating the mechanical foaming agent prior to removing the mechanical foaming agent from the breast prosthesis so as to aid in the removal of the mechanical foaming agent.

Additionally, in order to provide a more life-like appearance to the breast prosthesis the method includes a step of stippling a surface of the mold with a silicone material having a first, relatively dark pigmentation before forming the skin surface with a silicone material having a second, relatively light pigmentation.

In the following description there is shown and described a preferred embodiment of this invention, simply by way of illustration of one of the molds best suited to carry out the invention. As it would be realized, the invention is capable of other different embodiments and its several details are capable of modification in various, obvious aspects, all without departing from the invention. Accordingly, the drawings and descriptions would be regarded as illustrative in nature and not as restrictive.

### BRIEF DESCRIPTION OF THE DRAWING

The accompanying drawing incorporated in and forming a part of the specification, illustrates several aspects of the present invention and together with the description serves to explain the principles of the invention. In the drawing:

FIG. 1 is a schematical cross-sectional view of a breast prosthesis constructed in accordance with the teachings of the present invention.

Reference will now be made in detail to the present preferred embodiment of the invention, an example of which is illustrated in the accompanying drawing.

### DETAILED DESCRIPTION OF THE INVENTION

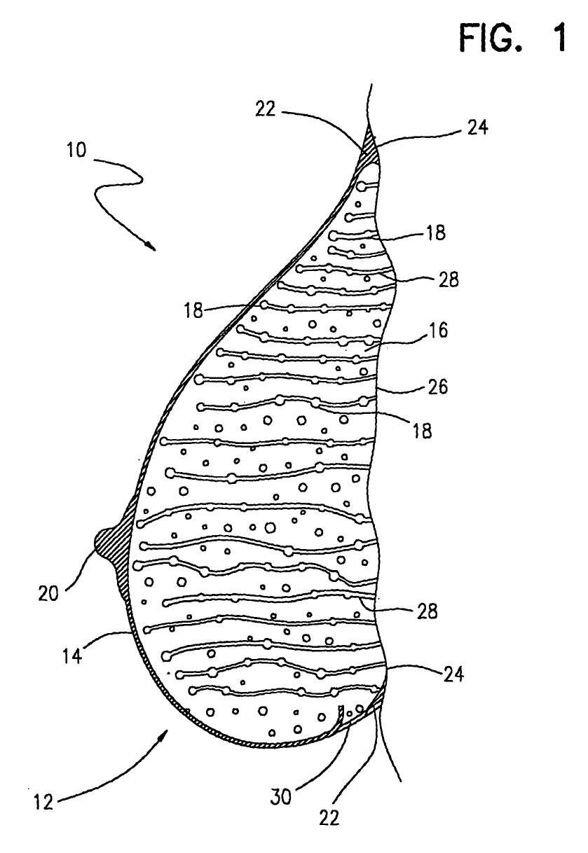

Reference is now made to FIG. 1 showing a medical prosthesis, in this instance, a breast prosthesis, generally designated by reference numeral 10. It should be appreciated, that the breast prosthesis 10 is only illustrative of one possible embodiment of the present invention and that other medical prosthesis of substantially any body part may be constructed in accordance with the teachings of the present invention.

As shown in FIG. 1, the breast prosthesis 10 includes a body, generally designated by reference 12 having a silicone skin 14 between about 1.0 to about 2.0 mm thick and a silicone foam body core 16 having from about 25 to about 41 and more typically about 33% by volume silicone and from about 75 to 59 and more typically about 67% by volume void space.

The silicone foam body core 16 includes a consistent cellular structure formed by a plurality of interconnected voids 18. Each void 18 has a volume of between about 0.0645 to about 268.032 mm and more typically a volume of between about 65.45 to about 179.5948 mm³. The silicone foam body core has a density of between about 5.0 to about 10.0 lbs/ft³.

The silicone skin 14 of the breast prosthesis 10 has a viscosity of about 50,000±10 CPS, a specific gravity of between about 1.0 and about 1.01, a hardness shore A of about 8 and about 12, a tear resistance of about 120 ppi, a tensile strength of about 475 psi, and an elongation of between about 550 to about 600% and a shrinkage of about 0.1%. The silicone foam body core 16 has a compression deflection at 25% psi of between about 0.5 to about 1.0 and at 50% psi of between 0.5 to about 3.0 and a specific gravity of between about 0.1 and about 0.15.

The unique physical characteristics provided by the combination of the silicone skin 14 and silicone foam body core 16 result in a breast prosthesis 10 with tactile properties more closely mimicking those of actual breast tissue than has heretofore been found possible to achieve in the art. Accordingly, the present invention represents a significant advance in this technology.

As further shown in FIG. 1, the breast prosthesis 10 includes a nipple 20 formed from silicone. Additionally, as shown in FIG. 2, the breast prosthesis includes a peripheral margin 22 of silicone about the rear wall of the breast prosthesis. This margin 22 is from about 1.0 to about 2.0 mm thick and from about 9.5 to about 19.1 mm wide. The margin 22 provides a rear surface 24 which is capable of receiving an appropriate adhesive for securing the breast prosthesis 10 to the dermis of the chest wall of the patient for which the prosthesis has been made. As should be appreciated, the rear wall 26 may be molded to provide a contour matching the dermis and chest wall of the patient so that the breast prosthesis 10 may be locked in place and secured more readily in the desired position.

It should also be appreciated that the rear wall 26 may be formed from barrier silicone if desired at a thickness of about 1.0 to about 2.0 mm. This can aid in reducing any potential for skin irritation along the chest wall in the area of prosthesis attachment.

The production of the breast prosthesis 10 will now be described in detail. The method initially includes the step of making a mold of appropriate shape for the breast prosthesis. That mold may be made in any known manner including by actually molding or imaging an existing breast including the remaining breast of a single mastectomy patient for which the breast prosthesis 10 is being constructed. The molding process may also include the molding of the chest wall of the patient at the mastectomy site. This will allow the rear wall 26 of the breast prosthesis 10 to be molded with a matching contour which will aid in locking the breast prosthesis 10 in place on the chest wall in the manner previously described.

After making the mold, the method includes the forming of a nipple in the mold. More specifically, the nipple is formed by applying silicone on the mold to the nipple area. The nipple 20 is shaped to match the remaining nipple of the single mastectomy patient. Generally, the nipple is from about 2.0 to about 5.0 mm thick. The silicone and appropriate amount of polymerization catalyst is mixed with an appropriate pigment to provide the desired color to the nipple 20 and areola areas. After completing the application of the silicone to the nipple and areola areas, some stippling may be provided around the curved frontal section of the mold utilizing any extra silicone prepared and pigmented for creating the nipple and areola. Following nipple and areola construction and the completion of stippling, the silicone applied to the mold is allowed to cure for approximately 30 minutes. The time, of course, may be varied by adding an accelerant if desired. After the nipple and areola area and stippling have partially cured, the skin surface is formed. Specifically, a mixture of silicone, polymerization catalyst, and an appropriate amount and color of skin tone pigment is applied in a thin layer approximately 1.0 mm to about 2.0 mm thick along the curved front face portion of the mold. At the same time, the silicone is applied around the peripheral margin of the rear portion of the mold so as to form the surface that will receive the adhesive for mounting the breast prosthesis securely to the chest wall of the patient. The silicone is applied in the margin area to a thickness of about 1.0 to about 2.0 mm and at a width of about 0.375 to 0.75 inches wide. The silicone skin 14 and margin 22 are then allowed to partially cure for, for example, 60 minutes. The two mold sections are then joined before curing is completed so as to insure a seamless connection between the silicone skin 14 painted over the inner surface of the front section of the mold and the margin 22 painted over the inner surface of the rear section of the mold.

The rear section of the mold includes an opening for delivering the components of the silicone foam body core 16 to the interior of the mold. More specifically, an appropriate amount of silicone, polymerization catalyst and mechanical foaming agent are mixed and poured into the mold through this opening. Silicone fluid may also be added to the mixture at up to 40% by weight of the silicone but more typically around 10% by weight in order to lower the hardness of the silicone foam body core 16.

The mixture preferably includes from about 25 to about 41% by volume silicone and from about 75 to about 59% by volume void space. A mixture of about one part by volume silicone and about two parts by volume mechanical foaming agent has been found to be particularly effective in creating a breast prosthesis with tactile properties closely mimicking actual breast tissue. To achieve this goal the mechanical foaming agent comprises solid bodies that are insoluble in silicone. Each of these individual solid bodies has a volume of between about 0.06545 to about 268.0832 mm³ and more typically a volume of between about 65.45 to about 179.5948 mm³. The mechanical foaming agent produces relatively large volume voids that provide the breast implant 10 with life-like flexibility, resiliency and texture.

After adding the foam body core forming mixture to the mold, the foam body core 16 is allowed to fully cure for 24 hours. Since the skin 14 and margin 22 were only partially cured prior to adding the silicone, polymerization catalyst and mechanical foaming agent for the body core 16, the skin, margin and core cure to form a single, integral body 12. The mold is then opened and the breast prosthesis 10 is removed.

This is followed by the step of removing the mechanical foaming agent from the cured breast prosthesis. Removing may be accomplished in a number of ways including but not limited to (a) pressing the mechanical foaming agent from the breast prosthesis, (b) vacuuming the mechanical foaming agent from the breast prosthesis and (c) applying a magnetic field to draw the mechanical foaming agent from the breast prosthesis where the mechanical foaming agent is, for example, iron balls, susceptible to attraction via a magnetic field.

Specifically, each individual solid body is removed intact from the core 16 by passing through the rear wall 26. As each solid body is moved through the core 16, it cuts a channel or passageway 28 in the silicone material. Thus, it should be appreciated that each void 18 is connected with a first end of a passageway 28 that opens at a second end through the rear wall 26. Still further, it should be appreciated that each passageway 28, while present, is at least partially collapsed or closed by the surrounding resilient silicone material. Thus, the passageways 28 take the form of a series of randomly spaced discontinuities or rifts in the foam body core 16. In addition, many of the solid bodies move through the core during removal along paths of least resistance. As such, many of the voids 18 are interconnected by the passageways 28. The resulting unique structural arrangement plays a role in providing the desired tactile properties of the breast prosthesis of the present invention.

It should be appreciated that removal of the mechanical foaming agent may be aided by lubricating the mechanical foaming agent prior to attempting removal. This may be simply done by applying soapy water to the breast prosthesis so that the soap coats the mechanical foaming agent and makes it slippery and thereby more readily removable from the foam structure.

In accordance with an additional aspect of the present invention, it should be appreciated that the rear wall of the mold within the margin 22 may be formed from a thin layer of barrier silicone that when cured is between about 1.0 and 2.0 mm thick. The mechanical foaming agent may be removed from this silicone barrier layer by pressing through the wall thereof.

A breathing port or vent 30 may also be provided in the skin 14 adjacent the peripheral margin 22 along the lower side of the prosthesis 10. In this position, the breathing port 30 is substantially hidden from view. The port 30 allows air to flow through the foam body core 16 during compression of the prosthesis 10. The port 30 also allows water to flow in and out freely during swimming and other water activities. Still further, the port 30 allows the chest wall abutting the rear wall 26 of the prosthesis to breathe.

In summary, numerous benefits result from employing the concepts of the present invention. The prostheses of the present invention are characterized by a unique structure of interconnected voids that provide the prostheses with tactile properties that more closely mimic actual tissue in a manner not previously possible in the art. The prostheses may also include a vent 30 that allows the prostheses to breathe thereby increasing user comfort.

The foregoing description of a preferred embodiment of the invention has been presented for purposes of illustration and description. It is not intended to be exhaustive or to limit the invention to the precise form disclosed. Obvious modifications or variations are possible in light of the above teachings. For example, the mechanical foaming agent could be a crystalline or other solid material that is insoluble in silicone but soluble in a selected solvent that will not damage the silicone. Such a foaming agent could be removed after the silicone foam body core is cured by emersing the core in a solvent which dissolves the mechanical foaming agent and then washing that solvent from the breast prosthesis 10. While requiring extra solubilizing and drying steps, such a procedure could be utilized to produce a breast prosthesis with the relatively large voids described to provide improved tactile and tissue mimicking properties. It should be appreciated, however, that this approach would not interconnect the voids in the manner of the mechanical foaming agent described above which is removed as intact solid bodies. Thus, there are resulting structural differences in the two techniques which produce differing overall properties and characteristics such as tactile properties.

The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

What is claimed:

1.) A medical prosthesis, comprising:
a body having a silicone skin and a silicone foam body core.

2.) A medical prosthesis, comprising:
a body having a silicone skin between about 1.0 to about 2.0 mm thick and a silicone foam body core having about 25 to 41 percent by volume silicone and from about 75 to about 59 percent by volume void space.

3.) The medical prosthesis of claim 2 wherein said silicone foam body core includes a plurality of voids, each void of said plurality of voids having a volume between about 0.06545 to about 268.0832 mm³.

4.) The medical prosthesis of claim 2, wherein said silicone foam body core includes a plurality of voids, each void of said plurality of voids having a volume between about 65.45 and about 179.5948 mm³.

5.) The medical prosthesis of claim 2, wherein said silicone foam body core is about 33% by volume silicone and about 67% by volume void space.

6.) The medical prosthesis of claim 2, wherein said silicone foam has a density of between about 5.0 to about 10.0 lbs/ft³.

7.) The medical prosthesis of claim 2, wherein said skin has a viscosity of about 50,000+10 CPS, a specific gravity of between about 1.0 and 1.01, a hardness shore A of between about 8 and about 12, a tear resistance of about 120 PPI, a tensile strength of about 475 psi, an elongation of between about 550 to about 600 percent and a shrinkage of about 0.1 percent.

8.) The medical prosthesis of claim 2, wherein said silicone foam body core has a compression deflection of 25% psi between about 0.5 to about 1.0 and at 50% psi of between about 0.5 to about 3.0 and a specific gravity of between about 0.1 and about 0.15.

9.) A breast prosthesis, comprising; a body having a silicone skin and a silicone foam body core.

10.) The breast prosthesis of claim 9, wherein said skin has a thickness of between about 1.0 to about 2.0 mm and said silicone foam body core is from about 25 to about 41 percent by volume silicone and from about 75 to about 59 percent by volume void space.

11.) The breast prosthesis of claim 10, wherein said silicone foam body core includes a plurality of voids, each void of said plurality of voids having a volume between about 0.06545 to about 268.0832 mm³.

12.) The breast prosthesis of claim 10, wherein said silicone foam body core includes a plurality of voids, each void of said plurality of voids having a volume between about 65.45 and about 179.5948 mm³.

13.) The breast prosthesis of claim 10, wherein said silicone foam of said breast prosthesis is about 33% by volume silicone and about 67% by volume void space.

14.) The breast prosthesis of claim 10, wherein said silicone foam has a density of between about 5.0 to about 10.0 lbs/ft³.

15.) The breast prosthesis of claim 10, wherein said skin has a viscosity of about 50,000+10 CPS, a specific gravity of between about 1.0 and 1.01, a hardness shore A of between about 8 and about 12, a tear resistance of about 120 PPI, a tensile strength of about 475 psi, an elongation of between about 550 to about 600 percent and a shrinkage of about 0.1 percent.

16.) The breast prosthesis of claim 10, wherein said silicone foam body core has a compression deflection of 25% psi between about 0.5 to about 1.0 and at 50% psi of between about 0.5 to about 3.0 and a specific gravity of between about 0.1 and about 0.15.

17.) The breast prosthesis of claim 9, further including a nipple of silicone.

18.) The breast prosthesis of claim 17, wherein said nipple is between about 2.0% and about 5.0 mm thick.

19.) The breast prosthesis of claim 9, further including a peripheral margin of silicone from about 1.0 to about 2.0 mm thick and from about 9.5 to about 19.1 mm wide.

20.) The breast prosthesis of claim 9, further including a rear surface having a profile matching a chest wall of a patient for which said breast prosthesis is made.

21.) The breast prosthesis of claim 20, wherein said rear wall is formed from barrier silicone and is about 1.0 to about 2.0 mm thick.

22.) A method making a breast prosthesis for a patient, comprising;
making a mold of appropriate shape for said breast prosthesis;
forming a nipple in said mold;
forming a skin surface and mounting rim on said mold;
forming a foam body core in said mold in contact with said skin surface and said mounting rim;
removing said breast prosthesis from said mold.

23.) The method of claim 22 including using silicone to form said nipple, said skin surface and said mounting rim.

24.) The method of claim 22, including using silicone foam to form said foam body core.

25.) The method of claim 22, wherein forming said nipple includes applying silicone on said mold.

26.) The method of claim 22, wherein forming said skin surface and mounting rim includes applying silicone on said mold.

27.) The method of claim 22, wherein forming said foam body core includes putting a mixture of silicone, a polymerization catalyst and a mechanical foaming agent into said mold.

28.) The method of claim 27, further including mixing said silicone, said polymerization catalyst and said mechanical foaming agent together wherein said mixture includes about 1 part by volume silicone foam and about 2 parts by volume mechanical foaming agent.

29.) The method of claim 27, further including employing solid bodies that are insoluble in silicone as said mechanical foaming agent.

30.) The method of claim 27, including using individual solid bodies having a volume of between about 0.06545 to about 268.0832 mm³.

31.) The method of claim 27, including using individual solid bodies having a volume of between about 65.45 to about 179.5948 mm³.

32.) The method of claim 27, further including removing said mechanical foaming agent from said cured breast prosthesis.

33.) The method of claim 32, wherein said removing is accomplished by pressing said mechanical foaming agent from said breast prosthesis.

34.) The method of claim 32, wherein said removing is accomplished by vacuuming said mechanical foaming agent from said breast prosthesis.

35.) The method of claim 32, wherein said removing is accomplished by applying a magnetic field to draw said mechanical foaming agent from said breast prosthesis.

36.) The method of claim 27, further including lubricating said mechanical foaming agent prior to removing from said breast prosthesis.

37.) The method claim 22, further including stippling a surface of said mold with a silicone material having a first relatively dark pigmentation before forming said skin surface with a silicone material having a second, relatively light pigmentation.

38.) A medical prosthesis, comprising:
a body having a resilient skin and a resilient foam body core, said foam body core including a plurality of voids and a plurality of discontinuities.

39.) The medical prosthesis of claim 38, wherein said voids are connected to said discontinuities.

40.) The medical prosthesis of claim 38, wherein each of said discontinuities has a first end in communication with one of said voids and a second end extending through a wall of said prosthesis.

41.) The medical prosthesis of claim 40, wherein some of said discontinuities communicate with more than one of said voids.

42.) A breast prosthesis, comprising:
a body having a resilient skin and a resilient foam body core, said foam body core including a plurality of voids and a plurality of discontinuities.

43.) The breast prosthesis of claim 42, wherein said voids are connected to said discontinuities.

44.) The breast prosthesis of claim 42, wherein each of said discontinuities has a first end in communication with one of said voids and a second end extending through a wall of said prosthesis.

45.) The breast prosthesis of claim 44, wherein some of said discontinuities communicate with more than one of said voids.
